(19) United States
(12) Patent Application Publication
(10) Pub. No.: US 2023/0233592 A1
(43) Pub. Date: Jul. 27, 2023

(54) PHARMACEUTICAL COMPOSITION FOR THE CHEMICAL INHIBITION OF TGS1 IN THE THERAPEUTIC TREATMENT OF TELOMEROPATHIES

(71) Applicants: UNIVERSITÀ DEGLI STUDI DI ROMA "LA SAPIENZA", Rome (IT); UNIVERSITÀ DEGLI STUDI DI TRIESTE, Trieste (IT)

(72) Inventors: Grazia Daniela RAFFA, Rome (IT); Stefano CACCHIONE, Rome (IT); Stefan SCHOEFTNER, Trieste (IT)

(73) Assignees: UNIVERSITÀ DEGLI STUDI DI ROMA "LA SAPIENZA", Rome (IT); UNIVERSITÀ DEGLI STUDI DI TRIESTE, Trieste (IT)

(21) Appl. No.: 17/999,634

(22) PCT Filed: May 24, 2021
(86) PCT No.: PCT/IB2021/054484

§ 371 Date: Nov. 22, 2022

(30) Foreign Application Priority Data
May 27, 2020 (IT) 102020000012577

(51) Int. Cl. A61K 31/7076; A61P 43/00; G01N 33/50

(52) U.S. Cl. CPC A61K 31/7076; A61P 43/00; G01N 33/502; G01N 2333/91011

(57) ABSTRACT

The present invention relates to an inhibitor of the TGS1 enzyme and/or compositions comprising such inhibitor and one or more excipients for the therapeutic treatment of clinical conditions characterized and/or caused by telomeropathies.

### FIELD OF THE INVENTION

The present invention relates to an inhibitor of the TGS1 enzyme (trimethylguanosine synthase 1), in particular Sinefungin, to increase the dosage of telomerase RNA (TERC) and to promote an increase in telomere length. The invention further relates to a pharmaceutical composition comprising such inhibitor and one or more excipients.

Such inhibitor can be used for the therapeutic treatment under pathological conditions characterized and/or caused by an excessive shortening of telomeres (telomeropathies). The present invention further provides an in vitro method to increase the TERC dosage and to promote an increase in telomere length in cells and/or in tissues obtained from patients affected by the above-mentioned pathologies.

### STATE OF ART

The telomeropathies include a variety of genetic diseases caused by mutations in the genes codifying by proteins which adjust the stability of the telomers and activity of telomerase, the enzyme which keeps constant the length of the telomeres by protecting them from cellular senescence and apoptosis (Niewisch, M. R. & Savage, S. A. Expert Rev Hematol, 2019). The telomeropathies, thereamong congenital dyskeratosis (DC) aplastic anaemia, idiopathic pulmonary fibrosis, Hoyeraal Hreidarsson syndrome, are genetic diseases having in common a similar primary defect: excessively short telomeres and strong reduction in the replicative power of different types of staminal cells (Niewisch, M. R. & Savage, S. A. Expert Rev Hematol, 2019). The staminal cells of the hematopoietic line are particularly affected, with consequent development of anaemia and immunodeficiency. One of the main hopes of therapeutic treatment consists in identifying strategies which could counterbalance the causes of telomeric dysfunctionalities and promote the lengthening of telomeres in the patients' cells (Boyraz, B. et al. J. Clin Invest 126, 2016; Fok, W. C. et al. Blood 133, 2019). Currently there are no effective treatments which act directly on the causative factors of the pathology and transplant represents the only hope to alleviate the specific effects caused by the damage of tissues.

One of the main mechanisms in the pathogenesis of telomeropathies is represented by a low dosage of TERC, the RNA component of telomerase enzyme, which involves the reduction in its activity, with consequent progressive shortage of telomeres. The TERC deficit is caused by function loss and haploinsufficiency for the gene which codifies RNA TERC or by recessive mutations in the genes which codify for PARN and Dyskerin, two proteins essential for maturation and stability of RNA TERC. Mutations in these three genes are found at high frequency in DC patients. The characterization of the mutations in PARN and Dyskerin genes and TERC haploinsufficiency showed that even a slight reduction in the dosage of this RNA has very severe phenotypic consequences and involves a drastic shortening of telomeres (Armanios, M. & Blackburn, E. H. Nat Rev Genet 13, 2012).

A very promising potential is represented by the identification of mechanisms which increase the TERC dosage, with the purpose of counterbalancing the progressive shortening of the telomeres in the patients' cells. The interest towards the identification of new effective treatments is enormous. The treatments currently available for telomeropathies (for example the transplant of hematopoietic staminal cells, in case of diseases with bone marrow insufficiency, such as DC) do not act directly on the primary causative factor, that is the short telomeres. Specifically, compounds with recognized effectiveness are not known, which can be used to stimulate the telomeric lengthening in the treatment of patients with telomeropathies and aimed at counterbalancing the deficit caused by a reduced RNA dosage of telomerase.

The possibility of regenerating the telomeres in the patients' cells, to increase the replicative power thereof, represents an excellent therapeutic opportunity.

Sinefungin

Sinefungin is an inhibitor of several metyltransferases specific for the nucleic acids, which use Adenosyl Methionine (Ado-Met) as methyl group donor. The action mechanism consists in the competition with Ado-Met for the bond to the donor site of methyl groups existing on the enzyme (Schluckebier, G. et al. J Mol Biol 265, 1997). Several studies showed that Sinefungin has antimicrobial (Yadav M. K. et al. Biomed Res Int, 2014) and antiviral (Zhao Z. et al. BMC Bioinformatics 17, 2016; Hercik K. et al. Arch Virol 162, 2017) properties, the latter determined by the capability of this molecule to block the activity of metiltransferases which add methyl groups to the cap existing at the end 5′ of viral RNA (RNA guanine-N7 methyltransferase) (Pugh C. S. et al. J. Biol Chem 253, 1978; Zheng S. et al. J Biol Chem 281, 2006; Li J. et al. J Virol 81, 2007). The molecule showed even antifungal effectiveness, mediated by the inhibition of mRNA cap guanine-N7 methyltransferase and Ado-Met synthase enzymes (Zheng S. et al. Nucleic Acids Res 35, 2007). The enzymes involved in the cap modification pathway at 5′ are different in virus, in fungi and in mammals and Sinefungin inhibits ten times more effectively, the fungal cap methyltransferase enzyme with respect to the human ortholog (Chrebet G. L. et al. J Biomol Screen 10, 2005). Sinefungin showed inhibitory activity against the protozoans of the genus Leishmania (Bhattacharya A. et al. Mol Cell Biol 12, 1992), Trypanosoma (McNally K. P. & Agabian N. Mol Cell Biol 12, 1992), Plasmodium (Trager W. et al. Exp Parasitol 50, 1980) and on Entamoeba histolytica (Ferrante A. et al. Trans R Soc Trop Med Hyg 78, 1984). The treatment with Sinefungin increased the survival of mice infected with Toxoplasma gondii (Ferrante A. et al. C R Acad Sci III 306, 1988) and with various insulated of Leishmania (Avila J. L. Am J Trop Med Hyg 43, 1990; Paolantonacci P. et al. Antimicrob Agents Chemother 28, 1985), without showing clinically detectable toxic effects. However, nephrotoxic effects were detected in two studies with high doses, performed in goats (Zweygarth E. et al. Trop Med Parasitol 37, 1986) and dogs (Robert-Gero M. et al. NATO ASI Series book series (NSSA, volume 171) Leishmaniasis, 1989). Different analogous of Sinefungin (Devkota K. et al. ACS Med Chem Lett 5, 2014; Zheng W. Et al. J Am Chem Soc 134, 2012; Liu Q. et al. Bioorg Med Chem 25, 2017; Niitsuma M. et al. J Antibiot (Tokyo) 63, 2010; Tao Z. et al. Eur J Med Chem 157, 2018) were developed and tested in order to optimize the anti-parasitic properties thereof. Its similarity with S-adenosyl-methionine, makes Sinefungin a potential therapeutic adjuvant in homocystinuria, as kinetic stabilizer of cystathionine beta-synthase (Majtan T. et al. Biochimie 126, 2016). Moreover, the treatment with Sinefungin in a murine model for the renal fibrosis, determined an improvement in the pathology, through its inhibitory activity on SET7/9 lysine methyltransferase (Sasaki K. et al. J Am Soc Nephrol 27, 2016).

A study finalized to the characterization of cap methylation of the HIV viral transcripts demonstrates that in human cells Sinefungin inhibits RNA hypermethylase TGS1 (Yedavalli V. S. & Jeang K. T. Proc Natl Acad Sci 107, 2010). TGS1 trimethylates the cap of monomethylguanosine of various types of RNA transcripted by polymerasis II, thereamong snRNA, snoRNA, different viral RNA and RNA of telomerase. It was demonstrated that TGS1 is involved in RNA biogenesis of telomerase in S. cerevisiae and S. pombe (Franke J. Et al. J Cell Sci 121, 2008; Tang W. Et al. Nature 484, 2012). Yedavalli et al. demonstrate that the treatment with Sinefungin inhibits the nucleo-cytoplasmatic transportation of not spliced or partially spliced transcripts of HIV virus, by limiting the infective activity thereof (Yedavalli V. S. & Jeang K. T. Proc Natl Acad Sci 107, 2010).

### SUMMARY OF THE INVENTION

The role of Sinefungin as inhibitor of TGS1 was not further explored and assays in human cells were never carried out, aimed at testing the effect of Sinefungin on TERC or on other target RNA of TGS1. After extensive experimentation, the inventors found that RNA-hypermethylase TGS1 (Trimethylguanosine synthase 1), which trimethylates the cap of TERC monomethylguanosine is a negative regulator of the dosage of this RNA and mutations in TGS1 induce a considerable increase in the telomerase activity and lengthening of telomeres in cultured cells.

The invention is based upon the finding that the chemical inhibition of TGS1 enzyme in cultured human cells, by means of an inhibiting agent, stabilizes RNA TERC, by preventing degradation thereof and by determining an increase in the amount available for incorporation in telomerase and a consequent stimulation of telomerase activity, leading to a net lengthening of telomeres.

In particular, the inventors demonstrated that Sinefungin, an analogous of S-adenosyl-methionine, is an agent inhibiting the methyltransferase activity of TGS1, as suggested by preceding studies (Yedavalli V. S. & Jeang K. T. Proc Natl Acad Sci 107, 2010).

Such studies represent an absolute novelty in the field of the adjustment of telomerase biogenesis and demonstrate that the inhibition of TGS1 by genetic or chemical route, in particular by means of Sinefungin, determines an increase in the dosage of TERC, by detecting TGS1 as a therapeutic target for the pathologies caused by reduced activity of telomerase and excessive shortening of telomeres. In the light of the effects of such treatments, this finding has an enormous application potential in therapeutic field.

Therefore, a first aspect of the present invention is an inhibitor of the TGS1 enzyme, in particular Sinefungin, for use in the prevention and/or treatment of a pathology characterized and/or caused by telomeropathies. A second aspect of the present invention is a composition comprising an inhibitor of the TGS1 enzyme and one or more excipients. Thanks to its active components, the composition, the present invention relates to, allows to provide an improvement in the pathologies associated and/or caused by telomeropathies thanks to the effectiveness of the inhibitor of the TGS1 enzyme.

A third aspect of the present invention is an in vitro method to increase the dosage of telomerase RNA (TERC) and to promote an increase in telomere length in human cells and/or tissues. Said method comprises the insulation of cells and/or tissues obtained from patients affected by a pathology characterized and/or caused by telomeropathies, followed by the treatment of said cells and/or tissues with an inhibitor of the TGS1 enzyme or with a composition comprising said inhibitor and one or more excipients.

Other advantages and features of the present invention will result evident from the following detailed description.

### BRIEF DESCRIPTION OF FIGURES

FIG. 1.

Model illustrating the action of Sinefungin on the telomerase. TGS1 regulates negatively the abundance of the RNA component of telomerase, TERC. Sinefungin, by inhibiting TGS1, determines an increase in dosage of TERC, which results in an increase in the number of subunits of active telomerase and consequent lengthening of telomeres.

FIG. 2.

Sinefungin, analogous of S-adenosyl-methionine, inhibits the reaction of hypermethylation catalyzed by TGS1. (A) In vitro assay of hypermethylation performed by incubating 1 μg of recombinant GST-TGS1 or GST with 50 μM [³H—CH3]AdoMet (SAM) and with 5 mM m⁷GTP (MMG), in presence or not of 100 μM Sinefungin. Aliquots of the reaction mixture are placed on cellulose-polyethyleneimine and the reaction products are resolved by means of TLC. The incorporation of ³H—CHs in the methylated derivatives of MMg (DMG or TMG) is quantified by means of liquid scintillation counting. (B) Control reactions performed without protein or GST. (C) The entity of transfer of ³H-methyl to the dinucleotide cap is shown.

FIG. 3.

Sinefungin determines an increase in the dosage of hTR and a lengthening of telomeres. (A, E) qRT-PCR analysis of levels of hTR on RNA of UMUC3 cells (A) or of the cell line HeLa PARN KO (E), treated or not with 50 μM Sinefungin for 10 days. The bars represent the variation in the levels of hTR in the treated cells and in the not treated cells, obtained by three replicates.

(B,D,F) Determination of the telomeric length by means of Telomere Restriction Fragment analysis (TRF, performed according to the methods described in Roake, C. M. et al. Mol Cell 74, 2019) in two cell types characterized by short telomeres: the UMUC3 tumour cell line and the HeLa cells lacking in TGS1 or deadenylase PARN.

(B,D) TRF analysis was performed on genomic DNA extracted from TGS1-proficient (UMUC3 parental, TGS1 WT) or TGS1-deficient (TGS1 R1, TGS1 R2) UMUC3 cells, treated or not with Sinefungin in culture for the indicated period of time (all cell lines showed a doubling time comparable during the experiment). A lengthening of telomeres takes place in the treated control cells (compare lanes 1 and 2 in panels B-D) but not in the treated mutated clones TGS1 R1 and R2 (compare lane 4 against lane 5 and 7 against 8 in D). No lengthening of telomeres in the not-treated parental cells is observed (lanes 9-10).

(F) HeLa PARN KO cells were treated or not with 50 μM of Sinefungin for the period of time indicated in culture. Due to the reduced levels of RNA component of telomerase, the average telomeric length is shorter in the PARN KO cells (lane 13) rather than in the parental HeLa cell line.

(4.5 kb vs 7.5 kb). After 46 days of treatment with Sinefungin, a lengthening of telomeres in HeLa PARN KO cells (lanes 1 and 2) is noted.

### DETAILED DESCRIPTION OF THE INVENTION

### Glossary

The terms used in the present description are as generally understood by the person skilled in the art, unless differently indicated.

Under the acronym TGS1 in the present description the Trimethylguanosine synthase 1 protein is designated, characterized by methyltransferase activity, that is the capability of transferring methyl groups from a donor molecule to an acceptor. More specifically TGS1 relates to the human enzyme (see Uniprot Q96RS0 (TGS1_HUMAN)). Such enzyme is specific for the guanine (G) residue, for example it is involved in trimethylation of cap of monomethylguanosine of various types of RNA transcripted by polymerase II, thereamong snRNA, snoRNA, different viral RNA and RNA of telomerase.

The acronym TERC, even known as TR, hTR or TER, in the present application indicates the RNA component of the telomerase enzyme complex (telomerase RNA component). The TERC component is even known as “mould region”, as in fact it acts as template for the elongation of telomeres effected by telomerase (reverse transcriptase). The nucleotide sequence of TERC, which mainly consists of residues of cytosine (C) and adenosine (A), is complementary to the species-specific telomere sequence, and thus promotes the pairing between the telomere end of a chromosome and the catalytic site of the enzymatic complex, by guiding the correct synthesis of telomeric DNA.

Under the general term “telomeropathies” in the present invention, all pathologies and/or syndromes are indicated which are characterized and/or caused by a shortening of telomeres. Such pathologies include all diseases which are caused by mutations in genes directly involved in the metabolism of telomeres, known as “primary telomeropathies”, those having similar symptoms, but they are caused by genes controlling DNA repair, known as “secondary telomeropathies” (Opresko, P. L. & Shay, J. W. Ageing Res Rev 33, 2017), but even all conditions and/or disorders for which it was demonstrated that the short telomeres represent a susceptibility factor (Armanios, M. Mutat Res 730, 2012), such as for example pulmonary emphysema (Stanley, S. E. et al. J Clin Invest 125, 2015). Under “average telomere length” (abbreviated as Itm) reference is made to the average length of the terminal regions of a chromosome, consisted of highly repeated DNA. Since such physical quantity is referred to sequences of double-stranded DNA, it is measured generally based upon the number of pairs of bases consisting said sequences (abbreviated as pb, or bp or bps). Often the size of such sequences requires the use of the abbreviation “kbp”, equal one thousand pairs of bases. The average telomere length varies between the different species. In human beings, the telomeres have an average length comprised between 12 and 15 kb at birth. The telomeres shorten quickly during childhood, and afterwards they reduce by about 0-100 bp every year during the adult age, with a speed varying based upon the type of cell, exposition to oxidative or psychological stress, and other factors including mutations in genes directly involved in the metabolism of telomeres, or in genes controlling DNA repair.

As mentioned above, an aspect of the present invention relates to an inhibitor of the TGS1 enzyme (Trimethylguanosine synthase 1), for use in the prevention and/or treatment of a pathology characterized and/or caused by telomeropathies. The TGS1 enzyme which trimethylates the cap of monomethylguanosine of TERC is a negative regulator of the dosage of this RNA, therefore the inhibition of TGS1 induces a considerable increase in the dosage of the RNA component of telomerase TERC and determines a lengthening of the telomeres in the human cells.

According to an aspect of the present invention, the inhibitor agent of the TGS1 enzyme is a competitive inhibitor of S-adenosyl methionine. Not limiting examples of inhibitor agents suitable to be used in the present invention can be selected from the compounds shown in Table 1.

TABLE 1

| Compound | Bibliographic reference |
|---|---|
| Sinefungin | |
| S-adenosyl-homocysteine (SAH) | Wu J C. et al. 1987, Biol |
| | Chem 262, 4778-4786 |
| A9145c or (6E)-6-[5-(6-Aminopurin- | Borchardt R T. et al. 1979 |
| 9-yl)-3,4-dihyidroxioxolan-2- | Biochem Biophys Res Comm |
| ilidene]-2,5-bis(azaniumil)hexanoate | 89, 3 |
| Cyclosinefungin | Yebra M J. et al. 1991, |
| | Journal of Antibiotics 44, 10 |
| 5′-S-(2-methylpropyl) adenosine | Yebra M J. et al. 1991, |
| (SIBA) | Journal of Antibiotics 44, 10 |
| 5′-S-(1-methylpropyl) adenosine | Yebra M J. et al. 1991, |
| (ISOSIBA) | Journal of Antibiotics 44, 10 |
| 5′-S-methylthio-methyl adenosine | Yebra M J. et al. 1991, |
| | Journal of Antibiotics 44, 10 |
| aza-S-adenosyl methionine | Hausmann S. et al. 2005, J of |
| | Biol Chem 280, 21, 20404-20412 |
| carbocyclic aza-S-adenosyl | Hausmann S. et al. 2005, J of |
| methionine | Biol Chem 280, 21, 20404-20412 |
| N-propyl Sinefungin | Zheng W. et al. 2012, JACS |
| | 134, 18004-18014 |
| N-benzyl Sinefungin | Zheng W. et al. 2012, JACS |
| | 134, 18004-18014 |
| N-methyl Sinefungin | Zheng W. et al. 2012, JACS |
| | 134, 18004-18014 |
| N-ethyl Sinefungin | Zheng W. et al. 2012, JACS |
| | 134, 18004-18014 |
| analogous cycloalkanes of | Quing L. et al. 2017 |
| Sinefungin, as 6′(S)-9-(5′,6′,7′- | Biorganic & Med Chem 25, |
| Deoxy-6′-amine-7′-cyclopropyl-β- | 4579-4594 |
| D-heptafuranoside-1′)adenine | |
| 6′-methylenamine Sinefungin (GMS) | Wu H. et al. 2016 Biochemical |
| | Journal 473, 3049-3063 |
| 6′-homoSinefungin (HSF) | Cai X. et al. 2019 eLife |
| | 8: e47110 |
| Benzoaxaborole AN5568 | Steketee P C. et al. 2018, |
| (SCYX-7158) | PLOS Neglected Tropical |
| | Diseases 12(5) |

The inhibitor of the TGS1 enzyme according to the present invention is preferably Sinefungin, inhibitor of the methyltransferase activity. Sinefungin is a natural nucleoside, analogous of S-adenosyl methionine, and it has the following structure:

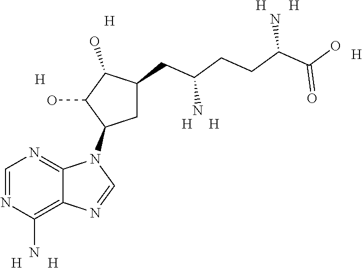

The present invention further relates to a composition comprising said inhibitor of the TGS1 enzyme according to one of the herein described embodiments and one or more excipients.

A not limiting example of composition according to the present invention comprises excipients selected from those usually known in the state of art such as diluents (for example dibasic calcium phosphate, lactose, microcrystalline cellulose and cellulose derivatives), absorbents, adsorbents, lubricants, binders, disintegrating agents, surfactants, antioxidants, preservatives, emulsifiers, moistening agents, chelating agents and mixtures thereof.

The composition according to the present invention can further include protective compounds which, in some cases, could ease transportation and/or specific release of inhibitor in the cells of interest. Such compound could include any pharmacological transportation system known in the field, for example biocompatible polymers, microparticle systems, liposomes, nanostructured materials, photosensitive capsules, nanoparticles, cationic lipids.

The administration routes of the composition of the present invention include, but they are not limited thereto: oral route, intra-arterial route, intranasal route, via intraperitoneal route, intravenous route, intramuscular route, subcutaneous route or transdermic route.

According to an aspect of the present invention, the increase in the average telomere length determined by the inhibitor of the TGS1 enzyme or by a composition comprising such inhibitor according to any one of the herein described formulations will be of at least 0.5 kb.

The present invention further relates to the use of the inhibitor of the TGS1 enzyme or of the compositions comprising said inhibitor according to any one of the herein described embodiments, in the prevention and/or treatment of all pathologies characterized by short telomeres, shown in Table 2.

Among these pathologies there are the diseases caused by mutations in genes directly involved in the metabolism of the telomeres (primary telomeropathies), or those with similar symptomatology, but caused by genes which control the DNA repair (secondary telomeropathies) (Opresko, P. L. & Shay, J. W. Ageing Res Rev 33, 2017). These categories include, but they are not limited thereto: aplastic anaemia, Coats' plus syndrome, dyskeratosis congenita, Hoyeraal Hreidarsson syndrome, acute leukemia, idiopathic pulmonary fibrosis, Revesz syndrome, ataxia telangiectasia, Bloom syndrome, Werner syndrome, RECQL4 disorders, Hutchinson-Gilford Progeria.

Other pathologies characterized by short telomeres include those conditions therefor it was demonstrated that the short telomeres represent a susceptibility factor (Armanios, M. Mutat Res 730, 2012): these include idiopathic pulmonary fibrosis, non-specific pulmonary pneumonitis, bronchiolitis obliterans organizing pneumonia, chronic hypersensitivity pneumonitis, interstitial fibrosis, pulmonary emphysema, pulmonary emphysema combined with pulmonary fibrosis, macrocytosis, cytopenias, bone marrow hypoplasia, bone marrow aplasia, myelodysplastic syndromes, acute myeloid leukemia, transaminase increase, atrophy, fibrosis, cryptogenetic cirrhosis.

TABLE 2
Clinic conditions characterized by short telomeres which could
benefit from treatment (modified by Armanios, M. Mutat Res 730,
2012; Stanley, S. E. et al. J Clin Invest 125, 2015).

| Primary telomeropathies | Secondary telomeropathies |
|---|---|
| aptastic anemia | [symbol] |
| [symbol] plus | [symbol] Syndrome |
| [symbol] | [symbol] Syndrome |
| Hoyeraal [symbol] syndrome | [symbol] disorders |
| acute leukemia | [symbol] |
| idiopathic pulmonary fibrosis | |
| R [symbol] syndrome | |

histologic and clinical pulmonary
manifestations associated with short telomeres

| | |
|---|---|
| Pulmonary disease | Bone marrow [symbol] |
| Idiopathic pulmonary fibrosis | Macrocytosis |
| (~65% of cases) | |
| Non-specific interstitial pneumonitis | [symbol] |
| (NSIP) | |
| Bronch [symbol] ob [symbol] organizing | Bone marrow |
| pneumonia | hypoplasia or aplasia |
| Chronic hypersensitivity pneumonitis | [symbol] syndromes |
| Interstitial fibrosis, non-classifiable | Acute myeloid leukemia |
| histology | |
| Emphysema alone or combined with | Liver disease |
| pulmonary fibrosis | |
| Pulmonary and extra-pulmonary | [symbol] atrophic liver |
| manifestations of telomere-mediated disease | |

[symbol]

indicates data missing or illegible when filed

An additional aspect of the present invention relates to the in vitro use of an inhibitor of the TGS1 enzyme to increase the dosage of telomerase RNA (TERC) and to promote an increase in telomere length in human cells and/or tissues.

An additional aspect of the invention is an in vitro method to increase the dosage of telomerase RNA (TERC) and to promote an increase in telomere length in human cells and/or tissues, said method comprising a treatment step of cultured cells and/or tissues with an inhibitor of the TGS1 enzyme or with a composition comprising said inhibitor and one or more excipients, wherein said cells and/or said tissues are obtained from patients suffering from a pathology characterized and/or caused by telomeropathies.

Not limiting specific examples of cells which can be treated in vitro with the method according to the present invention include epithelial cells, endothelial cells, nervous system cells, blood cells, immune system cells, keratinocytes, fibroblasts or myoblasts. The cells treated according to the in vitro method of the present application, could include tumour cells and/or non-tumour cells. In an aspect of the present invention the treated cells preferably are induced pluripotent stem cells and/or cells used to produce induced pluripotent stem cells, since such cells are capable of differentiating in different cell lines.

The method described in the present application could be used for the in vitro treatment of cells used in several applications, thereamong autologous or heterologous cell therapy, tissue engineering, growth of artificial organs, generation of induced pluripotent staminal cells, or cell differentiation techniques.

The induced pluripotent staminal cells derived from patients could be treated with the inhibitor of TGS1 to obtain a source of autologous cells wherein the telomeres were brought back to an optimal length, with the purpose of increasing the transplant success. This strategy would allow to avoid the problems related to the donor compatibility which are frequently found in the transplants of allogenic staminal cells. Should the treatment reveal to be well tolerated at the organism level, it could constitute an alternative to the transplant, which would allow to improve the prognosis of the patients wherein the transplant is not feasible.

The concentrations of the inhibitor compound will be determined based upon the response of the particular cell type in suitable toxicological assays, aimed at evaluating the minimum dosages of the compound under examination, capable of producing a RNA TERC increase ≥1.5 fold after 1 week of treatment and without causing variations in the growth rate. The measurement of the related telomere lengthening will have to be evaluated after one month of treatment and a length increase ≥0.5 kb with respect to the not treated control cells will be considered significant.

For the in vitro treatment, the compound or the composition could be administered by using any technique comprised in the state of art in the field of cell biology, cell culture, tissue culture or the like. The treatment according to the method of the present invention could be performed one or more times based upon the wished percentage of telomere extension. In an aspect of the present invention the in vitro treatment of the cells and/or tissues could last no more than 96 hours, no more than 72 hours, no more than 48 hours, no more than 36 hours, no more than 24 hours, no more than 18 hours, no more than 12 hours, no more than 8 hours, or even shorter periods of time. According to an aspect of the present invention such method for in vitro use even includes (a) the extraction of genomic DNA from cultured cells and (b) the analysis of the average telomere length (Itm). Such analysis can be performed by means of “Telomere Restriction Fragment” (TRF).

An in vivo method is also herein described, comprising the steps of the in vitro method according to any one of the described embodiments and a preliminary step for obtaining cells and/or tissues from patients and/or a step after the re-infusion treatment of such treated cells.

The in vitro method according to any one of the embodiments of the present invention could further be used to evaluate and select alternative inhibitor compounds of TGS1 enzyme, potentially usable for the prevention and/or treatment of a pathology characterized and/or caused by telomeropathies.

Therefore, the present invention also relates to an in vitro screening method for the identification of a candidate compound for use in the prevention and/or treatment of a pathology characterized and/or caused by telomeropathies, comprising the steps of:

(i) determining the methyltransferase activity of the TGS1 enzyme in the presence and absence of said candidate compound;

(ii) treating cultured cells and/or tissues with said candidate compound wherein said cells and/or said tissues are characterized by telomeropathies;

(iii) analysing the average telomere length before and after said treatment step (ii), wherein an increase in the average telomere length after said treatment step indicates that said compound is suitable for use in the prevention and/or treatment of a pathology characterized and/or caused by telomeropathies.

According to an embodiment of the in vitro screening method of the present invention, said step (i) of determining the methyltransferase activity of TGS1 enzyme can be performed by means of hypermethylation assay.

According to an aspect of the invention, said hypermethylation assay comprises the steps of:

(a) contacting said TGS1 enzyme with a methyl-group donor compound and with a substrate, in presence or absence of said candidate compound;

(b) separating and quantifying the methylated derivatives of said substrate that are produced.

In a preferred embodiment of the in vitro screening method according to the present invention, said used TGS1 enzyme is a recombinant TGS1 enzyme fused to a GST tag, and immobilized on a solid support, such as, for example, glutathione beads, said methyl-group donor compound is [³H—CH₃]Adenosyl-methionine (Ado-Met), said substrate is m⁷GTP (MMG).

According to an aspect of the invention, in said step (b) of the hypermethylation assay, the separation of the produced methylated derivatives of said substrate can be performed by means of thin layer chromatography (TLC), whereas their quantification can be performed by means of counting in liquid scintillation.

In an embodiment of the in vitro screening method according to the present invention, said step (iii) of analysing the average telomere length can be performed by means of “Telomere Restriction Fragment” (TRF) after extraction of genome DNA from the cultured cells.

According to an aspect of the present invention, said in vitro screening method can further include an additional step of determining the dosage of RNA of telomerase, for example by means of qRT-PCR and Northern Blotting, subsequent to said treatment step (iii), wherein an increase in the RNA dosage of telomerase indicates that said compound is suitable for use in the prevention and/or treatment of a pathology characterized and/or caused by telomeropathies.

The in vitro screening method according to any one of the herein described embodiments can even include a step of determining the catalytic activity of telomerase, for example by means of “Telomere repeats amplification protocol” (TRAP), in the presence and absence of said candidate compound, wherein an increase in the catalytic activity of telomerase indicates that said compound is suitable for use in the prevention and/or treatment of a pathology characterized and/or caused by telomeropathies.

### Examples

In Vitro Studies

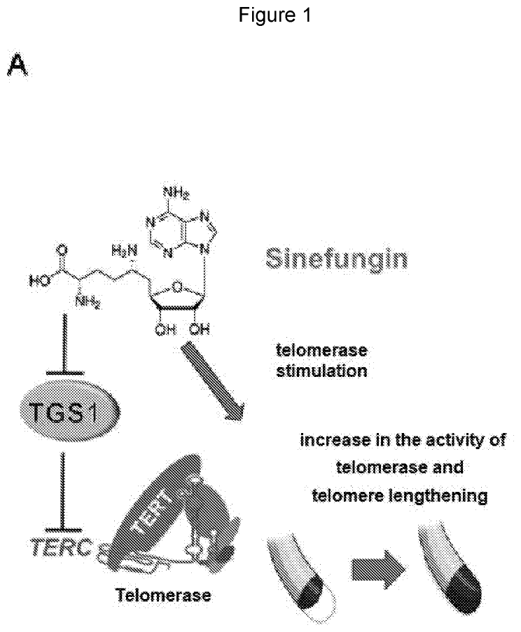

The identified mechanism, the present invention relates to, is illustrated in the model of FIG. 1. In the experiment shown in FIG. 3, it is demonstrated that Sinefungin is extremely effective in inducing the lengthening of telomeres. Sinefungin was administered to two cell lines with very short telomeres, already previously characterized: the mutant UMUC3 cells and HeLa cells for PARN deadenylase enzyme, one of the causative factor of DC; in the cells treated with Sinefungin, a significant lengthening of telomeres is noted.

In Vitro Hypermethylation Assay with Recombinant GST-TGS1 Enzyme

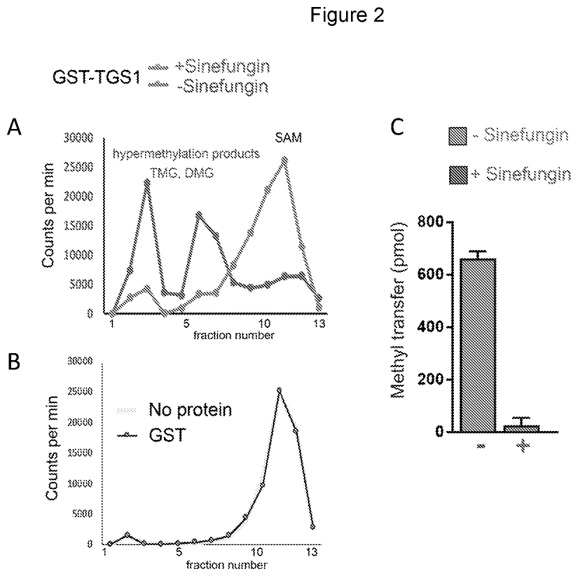

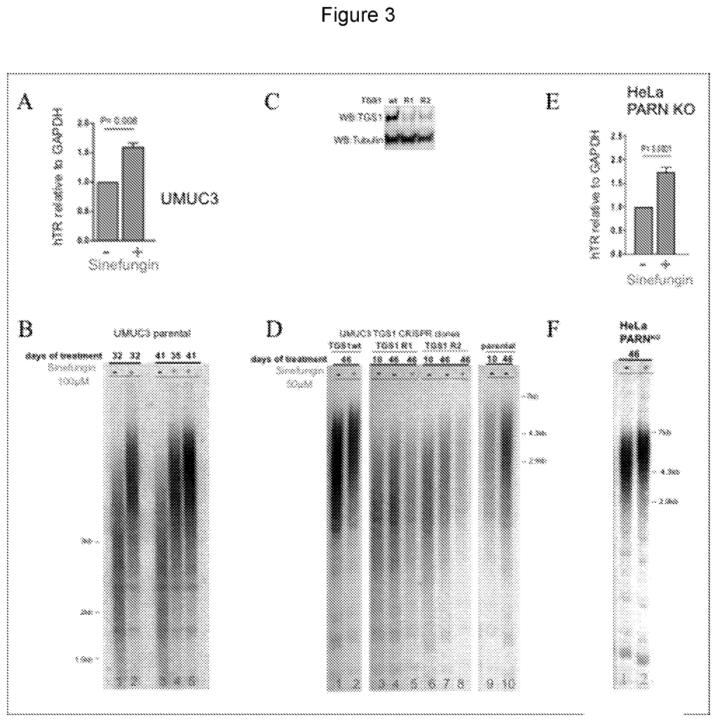

The Sinefungin capability of inhibiting the methyl-transferase activity of TGS1 enzyme was evaluated by means of recombinant in vitro hypermethylation assay by using recombinant TGS1 enzyme fused to protein GST. After having purified TGS1-GST from bacterial cells, still immobilized on glutathione beads, or GST alone, the assay was performed in presence or absence of Sinefungin, by using [³H—CH₃]AdoMet as methyl donor and m⁷GTP (MMG) as substrate. As shown in FIG. 2A, in the reaction mixtures containing the wild-type (WT) enzyme (GST-TGS1, blue line), two peaks were revealed much likely corresponding to the products of the methyl transfer on the m⁷GTP substrate which is converted into m^{2,7}GTP (DMG) and into m^{2,2,7}GTP (TMG). In the reactions not containing any protein, or in the reactions containing only GST beads (FIG. 2B), only one peak was revealed, likely corresponding to the chromatographic mobility of [³H—CH₃]AdoMet. When Sinefungin 100 μM was added to the reaction mixtures, only one peak was revealed co-migrating with [³H—CH₃]AdoMet (FIG. 2A, red line), to confirm the capability of Sinefungin to inhibit the methyl-transferase activity of TGS1 (FIG. 2C).

Treatment of UMUC3 Cells with Sinefungin

The effects of Sinefungin were tested on the tumour cell line of UMUC3 bladder, characterized by limiting levels of hTR for the activity telomerase and by short telomeres (Xu L. & Blackburn E. H. Mol Cell 28, 2007). The UMUC3 cells were treated with Sinefungin 100 μM for 10 days, and then the levels of RNA hTR were determined. The treated cells showed an increase in the levels of hTR equal to 1.5 times higher than that of the treated mutant cells (FIG. 3A), to indicate that the chemical inhibition of TGS1 has an effect on the dosage of hTR wholly comparable to the one induced by mutations in the TGS1 enzyme. In particular, a lengthening of the telomeres was observed when the UMUC3 cells were cultivated in presence of Sinefungin for over 15 population doublings (FIG. 3B). In order to confirm that Sinefungin is capable of striking specifically the TGS1 enzyme, a treatment with Sinefungin was tested on clones of UMUC3 cells, characterized by CRISPR-induced mutations in TGS1 (Chen et al.) (FIG. 3C), inside thereof there is a lengthening of the telomeres over time due to a deficiency of TGS1 (FIG. 3D). Control cells and mutant cells were cultivated for 46 days in presence or absence of Sinefungin. Contrary to the control cells, an additional lengthening of the telomeres was observed in the mutant UMUC3 cells for the TGS1 enzyme treated with Sinefungin (FIG. 3D, compare the lanes 4 and 5, 7 and 8). This observation demonstrates that the effect of Sinefungin on the telomere length is a consequence of TGS1 inactivation.

Treatment HeLa PARN KO Cells with Sinefungin

The effects of Sinefungin were tested on mutant HeLa cells for PARN deadenylase (PARN KO) enzyme, one of the causative factors of congenital dyskeratosis (Tummala et al., 2015) (Roake C. M. et al. Mol cell 74, 2019). PARN KO cells, obtained in the laboratory of S. Artandi (Stanford University), are characterized by short telomeres, due to the reduced levels of RNA component of telomerase. After 10 days of treatment with Sinefungin, a significative increase in the levels of hTR in PARN KO cells was observed (FIG. 3E). Moreover, as indicative factor of the treatment effectiveness with Sinefungin in cells characterized by reduced levels of hTR, a substantial increase in the telomere length in PARN KO cells was observed after 46 days of treatment with Sinefungin (FIG. 3F).

### CONCLUSIONS

The present invention is based upon the finding that the use of inhibitors in the methyltransferase activity of TGS1 enzyme, in particular Sinefungin, determines an increase in the dosage of RNA component of telomerase and promotes a lengthening of telomeres. Sinefungin is on the market, but it was never tested on human cells with the aim of stimulating telomerase and inducing lengthening of telomeres. In the herein described present invention the effect of inhibiting TGS1 on six different types of immortalized cells having tumour derivation occurred, by demonstrating the effectiveness thereof in the lengthening of telomeres.

In the light of such therapeutic effects, the present invention proposes an in vitro method to increase the dosage of telomerase RNA and to promote an increase in telomere length in human cells and/or tissues, derived from patients affected by pathologies characterized and/or caused by telomeropathies.

1. A method of preventing and/or treating a pathology characterized or caused by telomerophaties in a subject, comprising administering a therapeutically effective amount of an inhibitor of the TGSI enzyme to the subject in need thereof.

2. The method according to claim 1, wherein said inhibitor is a competitive inhibitor of Adenosyl-Methionine.

3. The method according to claim 1, wherein said inhibitor is selected from Sinefungin, S-adenosyl-homocysteine (SAH), A9145c, cyclosinefungin, 5′-S-(2-methylpropyl) adenosine (SIBA), 5′-S-(1-methylpropyl) adenosine (ISOSIBA), 5′-S-methylthio-methyl adenosine, aza-S-adenosyl-methionine, carbocyclic aza-S-adenosyl-methionine, N-methyl Sinefungin, N-ethyl Sinefungin, N-propyl Sinefungin, N-benzyl Sinefungin, 6′-methylenamine Sinefungin (GMS) or 6′-homoSinefungin (HSF), benzoaxaborole AN5568 (SCYX-7158), or analogous cycloalkanes of Sinefungin, such as 6′(S)-9-(5′,6′,7′-Deoxy-6′-amine-7′-cyclopropyl-□-D-heptafuranoside-1′) adenine.

4. The method according to claim 1, wherein said inhibitor is Sinefungin.

5. The method according to claim 1, wherein administration results in an increase in the amount of telomerase RNA (TERC) and an increase in telomere length.

6. The method according to claim 5, wherein administration results in an increase in the average telomere length of at least 0.5 kb.

7. The method according to claim 1, wherein said pathology is a primary and/or secondary telomeropathy.

8. The method according to claim 1, wherein said pathology is selected from aplastic anaemia, Coats' plus syndrome, dyskeratosis congenita, Hoyeraal Hreidarsson syndrome, acute leukemia, idiopathic pulmonary fibrosis, Revesz syndrome, ataxia telangiectapsia, Bloom syndrome, Werner syndrome, RECQL4 disorders, Hutchinson-Gilford progeria.

9. The method according to claim 1, wherein said pathology is selected from idiopathic pulmonary fibrosis, non-specific pulmonary pneumonitis, bronchiolitis obliterans organizing pneumonia, chronic hypersensitivity pneumonitis, interstitial fibrosis, pulmonary emphysema, pulmonary emphysema combined with pulmonary fibrosis, macrocytosis, cytopenias, bone marrow hypoplasia, bone marrow aplasia, myelodysplastic syndromes, acute myeloid leukemia, transaminase increase, atrophy, fibrosis, cryptogenetic cirrhosis.

10. A composition comprising an inhibitor of the TGS1 enzyme and one or more excipients.

11. The composition according to claim 10, wherein said inhibitor is a competitive inhibitor of adenosyl-methionine.

12. The composition according to claim 10, wherein said inhibitor is selected from Sinefungin, S-adenosyl-homocysteine (SAH), A9145c, cyclosinefungin, 5′-S-(2-methylpropyl) adenosine (SIBA), 5′-S-(1-methylpropyl) adenosine (ISOSIBA), 5′-S-methylthio-methyl adenosine, aza-S-adenosyl-methionine, carbocyclic aza-S-adenosyl-methionine, N-methyl Sinefungin, N-ethyl Sinefungin, N-propyl Sinefungin, N-benzyl Sinefungin, 6′-methylenamine Sinefungin (GMS) or 6′-homoSinefungin (HSF), benzoaxaborole AN5568 (SCYX-7158), or analogous cycloalkanes of Sinefungin, such as 6′(S)-9-(5′,6′,7′-Deoxy-6′-amine-7′-cyclopropyl-□-D-heptafuranoside-1′) adenine.

13. The composition for use according to claim 10, wherein said inhibitor is Sinefungin.

14.-17. (canceled)

18. The composition according to claim 10, wherein said composition is formulated for oral, intra-arterial, intranasal, intraperitoneal, intravenous, intramuscular, subcutaneous, or transdermal administration.

19. A method of increasing telomerase RNA (TERC) and telomere length in human cells and/or tissue in vitro, comprising contacting the human cells and/or tissue with an effective amount of an inhibitor of the TGS1 enzyme.

20. The method according to claim 19, wherein said inhibitor is selected from Sinefungin, S-adenosyl-homocysteine (SAH), A9145c, cyclosinefungin, 5′-S-(2-methylpropyl) adenosine (SIBA), 5′-S-(1-methylpropyl) adenosine (ISOSIBA), 5′-S-methylthio-methyl adenosine, aza-S-adenosyl-methionine, carbocyclic aza-S-adenosyl-methionine, N-methyl Sinefungin, N-ethyl Sinefungin, N-propyl Sinefungin, N-benzyl Sinefungin, 6′-methylenamine Sinefungin (GMS) or 6′-homoSinefungin (HSF), benzoaxaborole AN5568 (SCYX-7158), or analogous cycloalkanes of Sinefungin, such as 6′(S)-9-(5′,6′,7′-Deoxy-6′-amine-7′-cyclopropyl.

21. The method of claim 19, wherein said cells and/or said tissues are obtained from patients suffering from a pathology characterized and/or caused by telomeropathies.

22.-24. (canceled)

25. The method according to claim 21, wherein said pathology is selected from idiopathic pulmonary fibrosis, non-specific pulmonary pneumonitis, bronchiolitis obliterans organizing pneumonia, chronic hypersensitivity pneumonitis, interstitial fibrosis, pulmonary emphysema, pulmonary emphysema combined with pulmonary fibrosis, macrocytosis, cytopenias, bone marrow hypoplasia, bone marrow aplasia, myelodysplastic syndromes, acute myeloid leukemia, transaminase increase, atrophy, fibrosis, cryptogenetic cirrhosis.

26. The method according to claim 21, wherein said cells are induced pluripotent stem cells and/or cells used to produce induced pluripotent stem cells.

27. The method according to claim 21, wherein said method comprises a further step of extracting genomic DNA from the treated cultured cells and analysis of the average telomere length.

28. An in vitro screening method for the identification of a candidate compound for use in the prevention and/or treatment of a pathology characterized and/or caused by telomeropathies, comprising the steps of:
determining the methyltransferase activity of the TGS1 enzyme in the presence and absence of said candidate compound;
(ii) treating cultured cells and/or tissues with said candidate compound wherein said cells and/or said tissues are characterized by telomeropathies;
(iii) analyzing the average telomere length before and after said treatment step (ii), where an increase in the average telomere length after said treatment step indicates that said compound is suitable for use in the prevention and/or treatment of a pathology characterized and/or caused by telomeropathies.

29. The in vitro screening method according to claim 28, wherein said step (i) is performed by hypermethylation assay.

30. The in vitro screening method according to claim 29, wherein said hypermethylation assay comprises the steps of:
(a) contacting said TGS1 enzyme with a methyl-group donor compound and with a substrate, in the presence or absence of said candidate compound; and
(b) separating and quantifying the methylated derivatives of said substrate that are produced.

31. The in vitro screening method according to claim 28, wherein said TGS1 enzyme is a recombinant TGS1 enzyme fused to a GST tag.

32. The in vitro screening method according to claim 31, wherein said recombinant TGS1-GST enzyme is immobilized onto glutathione beads.

33. The in vitro screening method according to claim 30, wherein said TGS1 enzyme is a recombinant TGS1 enzyme fused to a GST tag and immobilized onto glutathione beads, said methyl-group donor compound is [³H—CH₃] adenosyl-methionine, said substrate is m⁷GTP (MMG).

34. The in vitro screening method according to claim 33, wherein in said step (b) said separation is carried out by thin layer chromatography (TLC) and said quantification is carried out by liquid scintillation counting.
